(19) United States
(12) Patent Application Publication
(10) Pub. No.: US 2026/0199211 A1
(43) Pub. Date: Jul. 16, 2026

(54) COSMETIC COMPOSITION, COSMETIC COMPOSITION FOR DISCOLORATION INHIBITION, AND DISCOLORATION INHIBITOR

(71) Applicant: SHISEIDO COMPANY, LTD., Tokyo (JP)

(72) Inventor: Yukako SATO, Tokyo (JP)

(21) Appl. No.: 19/131,681

(22) PCT Filed: Dec. 4, 2023
(86) PCT No.: PCT/JP2023/043202

§ 371 Date: May 21, 2025

(30) Foreign Application Priority Data
Dec. 21, 2022 (JP) 2022-204817

(51) Int. Cl. A61K 8/55; A61K 8/368; A61Q 19/02

(52) U.S. Cl. CPC A61K 8/55; A61K 8/368; A61Q 19/02

(57) ABSTRACT

A cosmetic composition is provided. The cosmetic composition includes phytic acid, a salt of phytic acid, or both, and alkoxysalicylic acid represented by a general formula (1) below, a salt of the alkoxysalicylic acid or both,

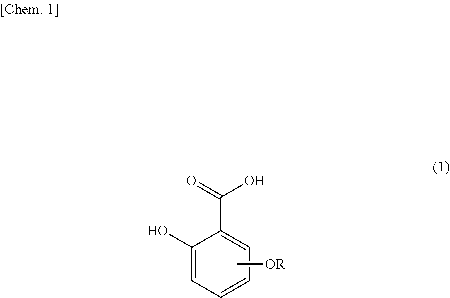

where, in the general formula (1), R is an alkyl group having a carbon number of 1 to 6.

### TECHNICAL FIELD

The present invention relates to cosmetic compositions, cosmetic compositions for discoloration inhibition, and discoloration inhibitors.

### BACKGROUND ART

It has been reported that alkoxysalicylic acid or a salt of alkoxysalicylic acid has an inhibition action for melanin production, and a topical skin preparation including the alkoxysalicylic acid or the salt of the alkoxysalicylic acid exhibits an excellent whitening effect (see, for example, Patent Document 1). However, when a composition including alkoxysalicylic acid is stored for a long period under severe conditions, discoloration may occur.

As a method of inhibiting discoloration of alkoxysalicylic acid, use of the alkoxysalicylic acid in combination with an inorganic salt, such as sodium pyrosulfite, has been known. However, sodium pyrosulfite may release an unpleasant odor, and therefore a blended amount of sodium pyrosulfite is limited, particularly in the case of a fragrance-free product.

Thus, the following technique has been reported. In the reported technique, phenol sulfonate is used in combination with sulfite, pyrosulfite, and vitamin C or a vitamin C derivative so that a blended amount of sodium pyrosulfite can be reduced (see, for example, Patent Document 2).

### CITATION LIST

### Patent Document

Patent Document 1: Japanese Unexamined Patent Application Publication No. H06-40886
Patent Document 2: Japanese Unexamined Patent Application Publication No. 2022-158488

### SUMMARY OF THE INVENTION

### Technical Problem

The present invention aims to provide a cosmetic composition that can inhibit discoloration of alkoxysalicylic acid, a salt of the alkoxysalicylic acid, or both, and has excellent storage stability.

### Solution to the Problem

The present inventors have conducted intensive studies to achieve the above object, and as a result, have found that phytic acid or a salt of phytic acid, which is a plant-derived component, can inhibit discoloration of alkoxysalicylic acid or a salt of alkoxysalicylic acid, and therefore a cosmetic composition having excellent storage stability can be provided. The present invention has been completed based on the above insight of the present inventors.

The cosmetic composition of the present invention, as the means of solving the above problems, includes phytic acid, a salt of phytic acid, or both, and alkoxysalicylic acid represented by the following general formula (1), a salt of the alkoxysalicylic acid, or both.

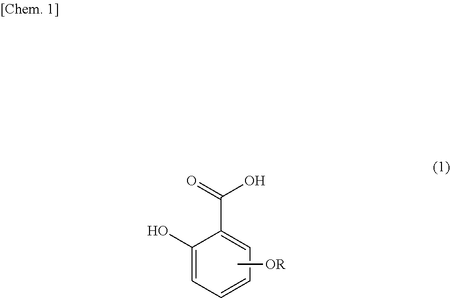

In the general formula (1), R is an alkyl group having a carbon number of 1 to 6.

### Effects of the Invention

According to the present invention, a cosmetic composition, which can inhibit discoloration of alkoxysalicylic acid, a salt of alkoxysalicylic acid, or both, and has excellent storage stability, can be provided.

### BRIEF DESCRIPTION OF THE DRAWINGS

FIG. 1 is a diagram presenting evaluation results of photostability of the cosmetic compositions of Examples 1 to 3 determined by xenon light irradiation.

FIG. 2 is a diagram presenting appearances of the cosmetic compositions of Examples 1 to 3 before light irradiation (controls).

### DETAILED DESCRIPTION OF THE EMBODIMENTS

### (Cosmetic Composition)

The cosmetic composition of the present invention includes (A) phytic acid, a salt of phytic acid, or both, and (B) alkoxysalicylic acid represented by the following general formula (1), a salt of the alkoxysalicylic acid, or both, and may further include other components, such as components typically used for cosmetics, as necessary.

### <Component (A): Phytic Acid and Salt of Phytic Acid>

The component (A) includes phytic acid, a salt of phytic acid, or both.

The phytic acid is also referred to as myo-inositol 1,2,3,4,5,6-hexakisphosphate (inositol hexaphosphate (IP₆)), and is a 6-fold phosphate ester of myo-inositol.

The phytic acid or the salt of phytic acid is a type of a biological substance. It has been known that phytin (water insoluble), which is a mixture of calcium phytate and magnesium phytate, or phytic acid is a major phosphorus storage form existing in many plant tissues, such as seeds.

Since the component (A) is a plant-derived component, the component (A) is preferable from the viewpoint of sustainability, which is a global goal.

The salt of phytic acid is not particularly limited as long as the salt is a physiologically acceptable salt, and may be appropriately selected according to the intended purpose. Examples of the salt include: alkali metal salts, such as sodium salts, potassium salts, and lithium salts; ammonium salts; amino acid salts; and the like. Among the above salts, a potassium salt is preferred.

As the component (A), a single substance may be used, or two or more substances may be used in combination.

The amount of the component (A) is not particularly limited, and may be appropriately selected according to the intended purpose. From the viewpoint that a discoloration inhibition effect of inhibiting discoloration of an alkoxysalicylic acid, a salt of alkoxysalicylic acid, or both is exhibited, and coloring of the phytic acid or the salt of phytic acid itself can be inhibited, the amount of the component (A) is preferably from 0.02 percent by mass to 0.2 percent by mass, more preferably from 0.03 percent by mass to 0.15 percent by mass, and yet more preferably from 0.04 percent by mass to 0.12 percent by mass, relative to a total amount of the cosmetic composition.

### <Component (B): Alkoxysalicylic Acid and Salt of Alkoxysalicylic Acid>

The component (B) includes an alkoxysalicylic acid represented by the following general formula (1), a salt of the alkoxysalicylic acid represented by the following general formula (1), or both.

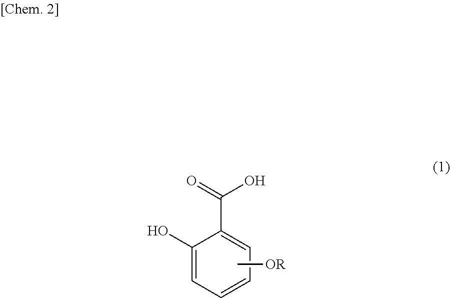

In the general formula (1), R is an alkyl group having a carbon number of 1 to 6.

The above R is an alkyl group having a carbon number of 1 to 6, which may have a straight chain or a branched chain. Examples of R include a methyl group, an ethyl group, an n-propyl group, an isopropyl group, an n-butyl group, an isobutyl group, a tert-butyl group, a sec-butyl group, and the like. Among the above alkyl groups, a methyl group or an ethyl group is preferred.

The position of the substituent OR is not particularly limited, and may be appropriately selected according to the intended purpose. The position of the substituent OR is preferably the 3-position, 4-position, or 5-position of salicylic acid, and more preferably the 4-position of salicylic acid.

Examples of the alkoxysalicylic acid include 3-methoxysalicylic acid, 3-ethoxysalicylic acid, 4-methoxysalicylic acid, 4-ethoxysalicylic acid, 4-propoxysalicylic acid, 4-isopropoxysalicylic acid, 4-butoxysalicylic acid, 5-methoxysalicylic acid, 5-ethoxysalicylic acid, 5-propoxysalicylic acid, and the like. Among the above alkoxysalicylic acids, 4-methoxysalicylic acid is preferred.

The alkoxysalicylic acid is a known substance, and can be readily synthesized according to a method known in the related art.

The salt of the alkoxysalicylic acid is not particularly limited as long as the salt is a physiologically acceptable salt, and may be appropriately selected according to the intended purpose. Examples of the salt include: alkali metal salts, such as sodium salts, potassium salts, and lithium salts; alkaline earth metal salts, such as calcium salts and magnesium salts; ammonium salts; amino acid salts; and the like. Among the above salts, a potassium salt is preferred. From the viewpoint of a cosmetic effect, availability, and the like, potassium 4-methoxysalicylate is more preferred.

It has been known that alkoxysalicylic acid or a salt of alkoxysalicylic acid, such as the potassium 4-methoxysalicylate, inhibits activity of tyrosinase, which is involved in melanin synthesis in melanocytes, to thereby exhibit a whitening effect (see Patent Document 1: Japanese Unexamined Patent Application Publication No. H06-40886, etc.).

As the component (B), a single substance may be used, or two or more substances may be used in combination.

The amount of the component (B) is not particularly limited, and may be appropriately selected according to the intended purpose, such as the intended use or the balance with other components. From the viewpoint of exhibiting effects of the component (B), such as a whitening effect, the amount of the component (B) is preferably from 0.1 percent by mass to 5 percent by mass, and more preferably from 0.5 percent by mass to 3.5 percent by mass, relative to a total amount of the cosmetic composition.

### <Other Components>

In addition to the component (A) and the component (B), the cosmetic composition may appropriately include components typically used for cosmetic compositions, such as oily components, ultraviolet-ray absorbers, surfactants, moisturizing agents, thickeners, polyhydric alcohols, powder components, synthetic resin emulsions, amino acids, organic amines, pH control agents, antioxidants, preservatives, chelating agents, and the like, as necessary.

### <<Oily Component>>

The oily components are not particularly limited, and may be appropriately selected according to the intended purpose. Examples of the oily components include oil and fats, wax, hydrocarbon oil, higher fatty acids, higher alcohols, synthetic ester oil, silicone oil, silicones, and the like.

Examples of the oil and fats include: liquid oil and fats, such as avocado oil, camellia oil, evening primrose oil, turtle oil, macadamia nut oil, corn oil, mink oil, olive oil, rapeseed oil, egg yolk oil, sesame oil, persic oil, wheat germ oil, camellia kissi seed oil, castor oil, linseed oil, safflower oil, cottonseed oil, perilla oil, soybean oil, peanut oil, tea seed oil, Torreya nucifera seed oil, rice bran oil, China wood oil, Japanese tung oil, jojoba oil, germ oil, triglycerin, glycerol trioctanoate, and glycerol triisopalmitate; solid oil and fats, such as cocoa butter, coconut oil, horse fat, hydrogenated coconut oil, palm oil, tallow, mutton tallow, hydrogenated beef tallow, palm kernel oil, lard, beef bone fat, Japan wax kernel oil, hydrogenated oil, neatsfoot oil, Japan wax, and hydrogenated castor oil; and the like.

Examples of the wax include beeswax, candelilla wax, cotton wax, carnauba wax, bayberry wax, insect wax, spermaceti wax, montan wax, bran wax, lanolin, Ceiba pentandra wax, lanolin acetate, liquid lanolin, sugarcane wax, lanolin fatty acid isopropyl ester, hexyl laurate, reduced lanolin, jojoba wax, hard lanolin, shellac wax, POE lanolin alcohol ether, POE lanolin alcohol acetate, POE cholesterol ether, polyethylene glycol lanolin fatty acid, POE hydrogenated lanolin alcohol ether, and the like.

Examples of the hydrocarbon oil include liquid paraffin, ozokerite, squalane, pristane, paraffin, ceresin, squalene, vaseline, microcrystalline wax, and the like.

Examples of the higher fatty acids include lauric acid, myristic acid, palmitic acid, stearic acid, behenic acid (docosanoic acid), oleic acid, 12-hydroxystearic acid, undecylenic acid, tall oil fatty acid, isostearic acid, linoleic acid, linolenic acid, eicosapentaenoic acid (EPA), docosahexaenoic acid (DHA), and the like.

Examples of the higher alcohols include: straight-chain alcohols, such as lauryl alcohol, cetyl alcohol, stearyl alcohol, behenyl alcohol, myristyl alcohol, oleyl alcohol, and cetostearyl alcohol; branched-chain alcohols, such as monostearyl glycerin ether (batyl alcohol), 2-decyltetradecinol, lanolin alcohol, cholesterol, phytosterol, hexyldodecanol, isostearyl alcohol, and octyldodecanol; and the like.

Examples of the synthetic ester oil include isopropyl myristate, cetyl octanoate, octyldodecyl myristate, isopropyl palmitate, butyl stearate, hexyl laurate, myristyl myristate, decyl oleate, hexyldecyl dimethyloctanoate, cetyl lactate, myristyl lactate, lanolin acetate, isocetyl stearate, isocetyl isostearate, cholesteryl 12-hydroxystearate, ethylene glycol bis(2-ethylhexanoate), dipentaerythritol fatty acid esters, N-alkyl glycol monoisostearate, neopentyl glycol dicaprate, diisostearyl malate, glyceryl bis(2-heptylundecanoate), trimethylolpropane tris(2-ethylhexanoate), trimethylolpropane triisostearate, pentaerythrityl tetraethylhexanoate, glyceryl tris(2-ethylhexanoate), trimethylolpropane triisostearate, cetyl 2-ethylhexanoate, 2-ethylhexyl palmitate, glyceryl trimyristate, tris(2-heptylundecanoic) glyceride, castor oil-fatty acid methyl esters, oleic oil, acetoglyceride, 2-heptylundecyl palmitate, diisobutyl adipate, 2-octyldodecyl N-lauroyl-L-glutamate, bis(2-heptylundecyl) adipate, ethyl laurate, bis(2-ethylhexyl) sebacate, 2-hexyldecyl myristate, 2-hexyldecyl palmitate, 2-hexyldecyl adipate, diisopropyl sebacate, 2-ethylhexyl succinate, ethyl acetate, butyl acetate, amyl acetate, triethyl citrate, crotamiton (C₁₃H₁₇NO), and the like.

Examples of the silicone oil include: linear silicone oil, such as dimethyl polysiloxane, methylphenyl polysiloxane, and methyl hydrogen polysiloxane; cyclic silicone oil, such as octamethylcyclotetrasiloxane, decamethylcyclopentasiloxane, dodecamethylcyclohexasiloxane, and tetramethylcyclotetrasiloxane; alkyl-modified silicone oil, which is a fluid at ambient temperature, such as caprylyl methicone, caprylyl trimethicone, and stearyl methicone; fluorine-modified silicone oil, such as dimethylsiloxane-diphenylsiloxane-methyl(perfluoroalkyl)siloxane copolymers; and the like.

Examples of the silicones include: alkylsilicone wax or alkoxysilicone wax, such as stearyl-modified dimethicone and behenyl-modified dimethicone; silicone resins each having a three-dimensional network structure; silicone rubber; and the like.

### <<Ultraviolet-Ray Absorber>>

Examples of the ultraviolet-ray absorbers include: benzoic acid-based ultraviolet-ray absorbers, such as p-aminobenzoic acid, monoglycerol p-aminobenzoate, ethyl N,N-dipropoxy-p-aminobenzoate, and ethyl N,N-dimethyl-p-aminobenzoate; anthranilic acid-based ultraviolet-ray absorbers, such as homomenthyl-N-acetylanthranilate; salicylic acid-based ultraviolet-ray absorbers, such as amyl salicylate, monomentyl salicylate, octyl salicylate, phenyl salicylate, benzyl salicylate, and p-isopropanolphenyl salicylate; cinnamic acid-based ultraviolet-ray absorbers, such as octyl methoxycinnamate, glyceryl 2-ethylhexanoate di-p-methoxycinnamate, octyl cinnamate, ethyl 4-isopropylcinnamate, methyl 2,5-diisopropylcinnamate, ethyl 2,4-diisopropylcinnamate, methyl 2,4-diisopropylcinnamate, propyl p-methoxycinnamate, isopropyl p-methoxycinnamate, isoamyl p-methoxycinnamate, octyl p-methoxycinnamate (2-ethylhexyl-p-methoxycinnamate), 2-ethoxyethyl-p-methoxycinnamate, cyclohexyl p-methoxycinnamate, ethyl α-cyano-β-phenylcinnamate, 2-ethylhexyl-α-cyano-β-phenylcinnamate, glyceryl mono-2-ethylhexanoyl-di-p-methoxycinnamate, 3-methyl-4-[methylbis(trimethylsiloxy)silyl]butyl-3,4,5-trimethoxy cinnamate, and methyl bis(trimethylsiloxy)silylisopentyl trimethoxycinnamate; benzophenone-based ultraviolet-ray absorbers, such as 2,4-dihydroxybenzophenone, 2,2′-dihydroxy-4-methoxybenzophenone, 2,2′,4,4′-tetrahydroxybenzophenone, 2-hydroxy-4-methoxybenzophenone, a salt of 2-hydroxy-4-methoxybenzophenone-5-sulfonic acid, 4-phenylbenzophenone, and 2-ethylhexyl 4′-phenylbenzophenone-2-carboxylate; 3-(4′-methylbenzylidene)-d,l-camphor, 3-benzylidene-d,l-camphor, urocanic acid, ethyl urocanate, 2,2′-hydroxy-5-methylphenylbenzotriazole, 2-(2′-hydroxy-5′-t-octylphenyl)benzotriazole, dibenzalazine, dianisoyl methane, 4-methoxy-4′-t-butyldibenzoylmethane, 5-(3,3-dimethyl-2-norbornylidene)-3-pentan-2-one, dimorpholinopyridazinone, and the like.

### <<Surfactant>>

The surfactants are not particularly limited, and may be appropriately selected according to the intended purpose. Examples of the surfactants include lipophilic nonionic surfactants, hydrophilic nonionic surfactants, anionic surfactants, cationic surfactants, amphoteric surfactants, and the like.

Examples of the lipophilic nonionic surfactants include: sorbitan fatty acid esters, such as sorbitan monooleate, sorbitan monoisostearate, sorbitan monolaurate, sorbitan monopalmitate, sorbitan monostearate, sorbitan sesquioleate, and diglyceryl sorbitan penta-2-ethylhexanoate; esters of glycerin or polyglycerin, such as glyceryl monostearate, glyceryl α,α′-oleate pyroglutamate, and glyceryl monostearate/malate; propylene glycol fatty acid esters, such as propylene glycol monostearate; hydrogenated castor oil derivatives; glycerin alkyl ethers; and the like.

Examples of the hydrophilic nonionic surfactants include: polyoxyethylene sorbitan fatty acid esters, such as polyoxyethylene sorbitan monooleate and polyoxyethylene sorbitan monostearate; polyoxyethylene sorbitol fatty acid esters, such as polyoxyethylene sorbitol monolaurate, polyoxyethylene sorbitol monooleate, and polyoxyethylene sorbitol monostearate; polyoxyethylene fatty acid esters, such as polyoxyethylene glyceryl fatty acid esters (e.g., polyoxyethylene glyceryl monostearate, polyoxyethylene glyceryl ronoisostearate, and polyoxyethylene glyceryl trilsostearate), polyoxyethylene monooleate, and polyoxyethylene distearate; polyoxyethylene alkyl ethers, such as polyoxyethylene lauryl ether, polyoxyethylene oleyl ethers, polyoxyethylene stearyl ether, polyoxyethylene 2-octyldodecyl ether, and polyoxyethylene cholestanol ether; polyoxyethylene alkylphenyl ethers, such as polyoxyethylene octylphenyl ether, polyoxyethylene nonylphenyl ether, and polyoxyethylene dinonylphenyl ether; Pluronic-type surfactants, such as Pluronic (registered trademark); polyoxyethylene polyoxypropylene alkyl ethers, such as polyoxyethylene polyoxypropylene cetyl ether, polyoxyethylene polyoxypropylene 2-decyltetradecyl ether, polyoxyethylene polyoxypropylene monobutyl ether, polyoxyethylene polyoxypropylene, hydrogenated lanolin, polyoxyethylene polyoxypropylene glyceryl ether, and PPG-13 decyltetradeceth-24; tetra(polyoxyethylene/polyoxypropylene) ethylenediamine condensates, such as tetronic; polyoxyethylene castor oil derivatives or polyoxyethylene hydrogenated castor oil derivatives, such as polyoxyethylene castor oil, polyoxyethylene hydrogenated castor oil, polyoxyethylene hydrogenated castor oil monostearate, polyoxyethylene hydrogenated castor oil triisostearate, polyoxyethylene hydrogenated castor oil monopyroglutamate monoisostearate, and polyoxyethylene hydrogenated castor oil maleate; alkanol amides, such as coconut oil fatty acid diethanolamide, lauric acid monoethanolamide, and fatty acid isopropanolamide; others, such as polyoxyethylene propylene glyceryl fatty acid esters, polyoxyethylene alkylamines, polyoxyethylene fatty acid amides, sucrose fatty acid esters, polyoxyethylene nonylphenyl formaldehyde condensates, alkylethoxydimethylamine oxide, and trioleyl phosphate; and the like.

Examples of the anionic surfactants include: fatty acid soap, such as sodium laurate and sodium palmitate; higher alkyl sulfates, such as sodium lauryl sulfate and potassium lauryl sulfate; alkyl ether sulfates, such as triethanolamine polyoxyethylene lauryl ether sulfate and sodium lauryl polyoxyethylene ether sulfate; N-acylsarcosine, such as sodium lauroyl sarcosinate; higher fatty acid amide sulfonates, such as sodium N-myristoyl-N-methyl taurate, sodium N-stearoyl-N-methyl taurate, sodium methyl cocoyl taurate, and sodium methyl lauroyl taurate; salts of phosphoric acid esters, such as a sodium salt of polyoxyethylene oleyl ether phosphate and a sodium salt of polyoxyethylene stearyl ether phosphate; sulfosuccinates, such as sodium di-2-ethylhexylsulfosuccinate, sodium monolauroyl monoethanolamide polyoxyethylene sulfosuccinate, and sodium lauryl polypropylene glycol sulfosuccinate; alkyl benzene sulfonates, such as sodium linear-dodecyl benzene sulfonate and triethanolamine linear-dodecylbenzene sulfonate; N-acylglutamic acids, such as monosodium N-lauroyl glutamate and disodium N-stearoyl glutamate; higher fatty acid ester sulfate salts, such as sodium hydrogenated glyceryl cocoate sulfate; polyoxyethylene alkyl ether carboxylic acid salts, α-olefin sulfonic acid salts, higher fatty acid ester sulfonic acid salts, secondary alcohol sulfate salts, higher fatty acid alkylolamide sulfate salts, sodium lauroyl monoethanolamide succinate, N-palmitoyl aspartic acid, diethanolamine or triethanolamine, alkali salts of cocoyl hydrolyzed collagen, and the like.

Examples of the cationic surfactants include: alkyl trimethyl ammonium salts (e.g., stearyltrimethylammonium chloride and lauryltrimethylammonium chloride), and dialkyl dimethyl ammonium salts (e.g., distearyldimethylammonium chloride); alkyl pyridinium salts, such as poly(N,N′-dimethyl-3,5,-methylenepiperidinum) chloride and cetylpyridinium chloride; alkyl quaternary ammonium salts, alkyldimethylbenzyl ammonium salts, alkyl isoquinolinium salts, dialkyl morpholinium salts, polyoxyethylene alkylamines, alkylamine salts, polyamine fatty acid derivatives, amylalcohol fatty acid derivatives, benzalkonium chloride, benzethonium chloride, cationic polymers, β-N—N-dimethyl-N-ethylammonioethyl acrylate/vinylpyrrolidone chloride copolymers, and the like.

Examples of the amphoteric surfactants include: imidazoline-based amphoteric surfactants, such as sodium 2-undecyl-N,N,N,-(hydroxyethylcarboxymethyl)-2-imidazoline and disodium 1-carboxyethyloxy-2-cocoyl-2-imidazolinium hydroxide; betaine-based amphoteric surfactants, such as 2-heptadecyl-N-carboxymethyl-N-hydroxyethylimidazolinium betaine, lauryl dimethyl aminoacetic acid betaine, alkyl betaine, amide betaine, and sulfobetaine; and the like.

### <<Moisturizing Agent>>

Examples of the moisturizing agents include cholesteryl-12-hydroxystearate, sodium lactate, dl-pyrrolidone carboxylate, urea, diglycerin ethylene oxide/propylene oxide adducts, acidic mucopolysaccharides (e.g., chondroitin sulfate and hyaluronic acid), and the like.

### <<Thickener>>

Examples of the thickeners include gum arabic, carrageenan, tragacanth gum, quince seed (quince), casein, sodium casein, dextrin, gelatin, sodium alginate, methyl cellulose, ethyl cellulose, carboxymethyl cellulose, hydroxyethyl cellulose, hydroxypropyl cellulose, polyvinyl alcohol, sodium polyacrylate, carboxyvinyl polymers, guar gum, xanthan gum, magnesium aluminum silicate, bentonite, hectorite, and the like.

### <<Polyhydric Alcohol>>

Examples of the polyhydric alcohols include ethylene glycol, propylene glycol, dipropylene glycol, 1,3-butylene glycol, glycerin, erythritol, trimethylolpropane, pentaerythritol, sorbitol, maltitol, diglycerin, polyethylene glycol, and the like.

### <<Powder Component>>

Examples of the powder components include: inorganic powder, such as talc, kaolin, mica, sericite, phlogopite, synthetic phlogopite, magnesium carbonate, calcium carbonate, aluminum silicate, silica, zeolite, barium sulfate, calcined calcium sulfate (calcined gypsum), calcium phosphate, fluoroapatite, hydroxyapatite, ceramic powder, metal soap (e.g., zinc myristate, calcium palmitate, and aluminum stearate), and boron nitride; organic powder, such as polyamide resin powder (nylon powder), polyethylene powder, methyl polymethacrylate powder, polystyrene powder, styrene-acrylic acid copolymer resin powder, polymethylsilsesquioxane powder, and cellulose powder; inorganic pigments, such as titanium dioxide, zinc oxide, iron oxide (red iron oxide), iron titanate, yellow iron oxide, black iron oxide, carbon black, lower oxide of titanium, manganese violet, cobalt violet, chromium oxide, ultramarine, and Prussian blue; pearl pigments, such as titanium oxide-coated mica, titanium oxide-coated bismuth oxychloride, colored titanium oxide-coated mica, bismuth oxychloride, and fish scale flakes; metal powder pigments, such as aluminum powder; organic pigments (e.g., Red No. 201, Red No. 202, Orange No. 203, Yellow No. 205, Yellow No. 401, and Blue No. 404), organic pigments of zirconium, barium, aluminum lakes, and the like (e.g., Red No. 3, Yellow No. 4, Green No. 3, and Blue No. 1), natural dyes (e.g., chlorophyll and β-carotene), and the like.

### <<Synthetic Resin Emulsion>>

Examples of the synthetic resin emulsions include acrylic resin emulsions, polyvinyl acetate resin emulsions, and the like.

### <<Amino Acid>>

Examples of the amino acids include glycine, leucine, phenylalanine, tyrosine, aspartic acid, glutamic acid, sodium glutamate, arginine, histidine, lysine, cystine, cysteine, sodium acylsarcosinate (sodium lauroylsarcosinate), acyl glutamic acid salts, glutathione, pyrrolidonecarboxylic acid, and the like.

### <<Organic Amine>>

Examples of the organic amines include monoethanol amine, diethanol amine, triethanol amine, morpholine, triisopropanol amine, 2-amino-2-methyl-1,3-propanediol, 2-amino-2-methyl-1-propanol, and the like.

### <<pH Control Agent, Antioxidant, Preservative, Chelating Agent>>

Examples of the pH control agents include buffers, such as lactic acid-sodium lactate, citric acid-sodium citrate, succinic acid-sodium succinate, and the like.

Examples of the antioxidants include tocopherols, dibutylhydroxytoluene, butylhydroxyanisole, gallic acid esters, and the like.

Examples of the preservatives include phenoxyethanol, ethyl paraben, butyl paraben, and the like.

Examples of the chelating agents include 1-hydroxyethane-1,1-diphosphonic acid, tetrasodium 1-hydroxyethane-1,1-bisphosphoate, disodium ethylenediaminetetraacetate, trisodium ethylenediaminetetraacetate, tetrasodium ethylenediaminetetraacetate, sodium citrate, sodium polyphosphate, sodium metaphosphate, gluconic acid, phosphoric acid, citric acid, ascorbic acid, succinic acid, ethylenediaminetetraacetic acid, trisodium ethylenediaminehydroxyethyltriacetate, disodium ethylenediaminetetraacetate, sodium hexametaphosphate, and the like.

In addition to the above, drugs, such as caffeine, tannin, verapamil, tranexamic acid and tranexamic acid derivatives, licorice extract, glabridin, Acanthus Radix extract by hot water extraction, various crude drugs, tocopherol acetate, glycyrrhizic acid, and derivatives or salts of glycyrrhizic acid; other whitening agents, such as arbutin; sugars, such as glucose, fructose, mannose, sucrose, trehalose and the like can be appropriately blended. In particular, when vitamin E esters, such as tocopherol acetate, are blended, a whitening effect of the alkoxysalicylic acid or a salt of the alkoxysalicylic acid is enhanced.

### [Viscosity]

The viscosity of the cosmetic composition is not particularly limited, and may be appropriately selected according to the intended purpose, such as an embodiment of the cosmetic composition.

The viscosity of the cosmetic composition can be measured, for example, by a viscometer (DIGITAL VISMETRON VDA, produced by Shibaura Systems Co., Ltd.) at 25° C.

### [Container]

The cosmetic composition is preferably packaged in a container.

The container is preferably an airtight container. The material of the airtight container is not particularly limited, and a material having low oxygen permeability can be appropriately selected depending on the intended purpose. For example, the container is preferably a container using a metal-deposited resin, a metal-laminated resin, a metal, glass, or any combination of the foregoing.

The form of the cosmetic composition may be, for example, an emulsion composition, such as a water-in-oil emulsion composition and an oil-in-water emulsion composition, an oil composition, or an aqueous composition, and may be appropriately selected from a liquid form, a solid form, a cream form, a paste form, a gel form, and the like.

The cosmetic composition can be produced according to a common method without any particular limitation, in the case where the cosmetic composition is an emulsified cosmetic, the emulsification method is not particularly limited.

### [Use]

The use of the cosmetic composition of the present embodiment is not particularly limited, and may be appropriately selected according to the intended purpose. Examples of the use include: skin care products, such as mask compositions (e.g., eye masks and face masks), creams (e.g., skin creams, eye creams, and lip creams), foundations, and make-up base; make-up cosmetics, such as eye shadows, blushers, and lipsticks; personal hygiene products, such as sunscreens and insect repellents; and the like.

### (Cosmetic Composition for Discoloration Inhibition)

The cosmetic composition for discoloration inhibition of the present invention includes phytic acid, a salt of phytic acid, or both (component (A)), and alkoxysalicylic acid represented by the general formula (1), a salt of the alkoxysalicylic acid, or both (component (B)). The cosmetic composition for discoloration inhibition may further include other components, such as components typically used for cosmetics, as necessary.

The cosmetic composition for discoloration inhibition can inhibit discoloration of the cosmetic composition as well as inhibiting discoloration of the alkoxysalicylic acid, the salt of the alkoxysalicylic acid, or both.

The component (A), the component (B), and other components may be appropriately selected from the features described in association with the above-described cosmetic composition of the present invention.

### (Discoloration Inhibitor)

The discoloration inhibitor of the present invention includes, as an active ingredient, phytic acid, a salt of phytic acid, or both (component (A)), and is a discoloration inhibitor for inhibiting discoloration of alkoxysalicylic acid represented by the general formula (1), a salt of the alkoxysalicylic acid, or both.

The component (A) may be appropriately selected from the features described in association with the above-described cosmetic composition of the present invention.

### EXAMPLES

The present invention will be more specifically described through Examples hereinafter, but the present invention is not limited to the following Examples. In the description of Examples, values of ratios (% and parts) are on a mass basis unless otherwise specified.

### Example 1

A cosmetic composition of Example 1 was prepared according to the following formulation, and the cosmetic composition of Example 1 was evaluated according to the following evaluation methods.

### <Formulation>

| | |
|---|---|
| Citric acid | 0.02 percent by mass |
| Sodium citrate | 0.08 percent by mass |
| 1,3-Butylene glycol | 8.00 percent by mass |
| Phenoxyethanol | 0.50 percent by mass |
| Potassium 4-methoxysalicylate | 1.00 percent by mass |
| Phytic acid | 0.03 percent by mass |
| Ion-exchanged water: (balance) | 90.37 percent by mass |
| Total | 100.00 percent by mass |

### Examples 2 and 3

Cosmetic compositions of Examples 2 and 3 were prepared in the same manner as in Example 1, except that the amount of phytic acid, which was 0.03 percent by mass in Example 1, was changed to 0.05 percent by mass and 0.10 percent by mass, respectively, and the cosmetic compositions of Examples 2 and 3 were evaluated according to the following evaluation methods.

### Comparative Example 1

A cosmetic composition of Comparative Example 1 was prepared in the same manner as in Example 1, except that phytic acid was omitted. The cosmetic composition of Comparative Example 1 was evaluated according to the following evaluation method.

### Comparative Examples 2 and 3

Cosmetic compositions of Comparative Examples 2 and 3 were prepared in the same manner as in Example 1, except that 0.03 percent by mass of phytic acid was respectively replaced with 0.10 percent by mass of ethylenediaminetetraacetic acid (EDTA) and 0.10 percent by mass of sodium hexametaphosphate, both serving as a chelating agent. The cosmetic compositions of Comparative Examples 2 and 3 were evaluated according to the following evaluation methods.

### <Evaluations>

### [Evaluation of Photostability by Xenon Light Irradiation]

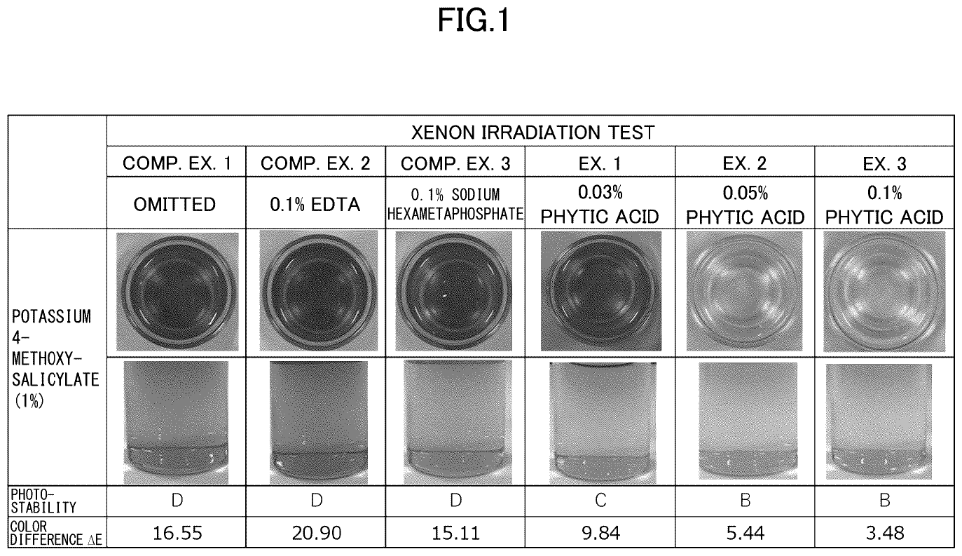

Each of the cosmetic compositions obtained as test samples was placed in a transparent airtight glass vessel. Xenon light irradiation (Xenon Weather Meter X25FL, produced by Suga Test Instruments Co., Ltd., irradiation condition: 30 MJ) was performed. The appearance of the test sample was observed with the naked eyes, and photostability of the cosmetic composition was evaluated based on the following evaluation criteria. The results are presented in FIG. 1.

### —Evaluation Criteria—

A: Discoloration of the cosmetic composition was not observed at all.
B: Minor discoloration of the cosmetic composition was observed, but was at an acceptable level.
C: Slight discoloration of the cosmetic composition was observed.
D: Obvious discoloration of the cosmetic composition was observed.

### [Measurement of Color Difference]

Moreover, the color difference (ΔE) of the test sample before and after the xenon light irradiation was determined by calculating a deviation of the measured value of the cosmetic composition after the xenon light irradiation from the measured value of the cosmetic composition just after the preparation (before light irradiation) using a spectrophotometer (Spectrophotometer SE7700, produced by Nippon Denshoku Industries Co., Ltd.). The results are presented in FIG. 1.

### [Appearance Before Light Irradiation]

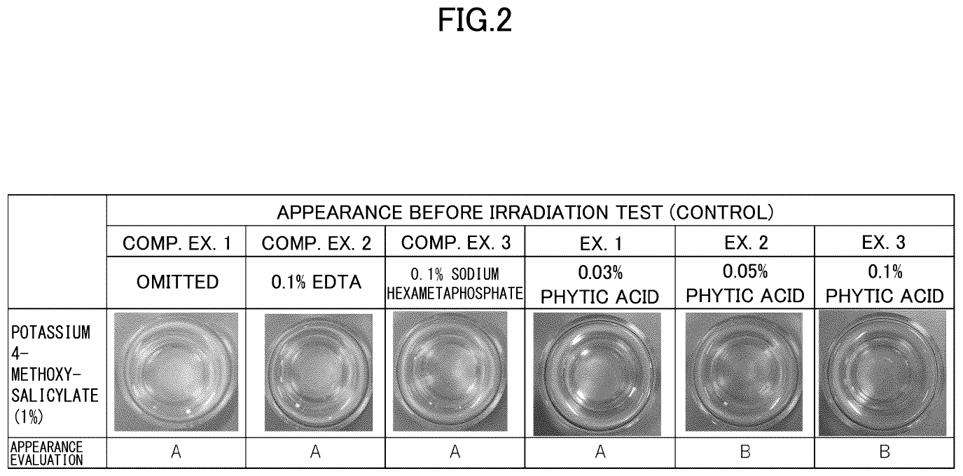

The appearance of the cosmetic composition before light irradiation was observed with the naked eyes in the same manner as in [Evaluation of photostability by xenon light irradiation], and the results were evaluated based on the above evaluation criteria. The results are presented in FIG. 2.

As a result, it was found that, when phytic acid was added in Examples 1 to 3, the cosmetic composition was slightly colored in brown due to the phytic acid itself (see FIG. 2), but discoloration of 4-methoxysalicylic acid was significantly inhibited under the high-power irradiation conditions of the xenon light irradiation and excellent storage stability was achieved compared with Comparative Examples 1 to 3. Conversely, obvious discoloration was observed in Comparative Example 1 in which phytic acid was not added, and in Comparative Examples 2 and 3, in which a different chelating agent was added instead of phytic acid.

It was found from the above results that the cosmetic compositions of Examples are suitably used as a cosmetic composition for discoloration inhibition, which inhibits discoloration of the alkoxysalicylic acid, a salt of the alkoxysalicylic acid, or both. Further, the discoloration inhibitor including, as an active ingredient, phytic acid, a salt of phytic acid, or both was suitably used as a discoloration inhibitor for inhibiting discoloration of the alkoxysalicylic acid, a salt of the alkoxysalicylic acid, or both.

As the cosmetic composition including phytic acid, a salt of phytic acid, or both, and alkoxysalicylic acid, a salt of alkoxysalicylic acid, or both, a lotion was prepared according to the following formulation.

### <Formulation of Lotion>

Ethanol: 4.00 percent by mass
Concentrated glycerin: 7.00 percent by mass
Diglycerin: 0.50 percent by mass
Dipropylene glycol: 8.00 percent by mass
1,3-Butylene glycol: 1.00 percent by mass
Carboxyvinyl polymer: 0.14 percent by mass
Xanthan gum: 0.04 percent by mass
Polyoxyethylene polyoxypropylene decyltetradecyl ether: 0.20 percent by mass
Potassium hydroxide: 0.085 percent by mass
Potassium 4-methoxysalicylate: 1.00 percent by mass
Dipotassium glycyrrhizate: 0.05 percent by mass
2-O-ethyl-L-ascorbic acid: 0.03 percent by mass
Magnesium L-ascorbyl phosphate: 0.001 percent by mass
Phytic acid: 0.10 percent by mass
Citric acid: appropriate amount
Sodium citrate: appropriate amount
Phenoxyethanol: appropriate amount
Red No. 227: appropriate amount
Yellow No. 4: appropriate amount
Fragrance: appropriate amount
Ion-exchanged water: balance
Total: 100.00 percent by mass

Although the present invention has been described through specific embodiments and Examples, the above embodiments and Examples are merely presented as examples, and the present invention is not limited to the above embodiments and Examples. Various changes, modifications, substitutions, omission, additions, combination, and the like are possible within the scope of the disclosure of the present invention.

The present application is based on and claims priority to Japanese Patent Application No. 2022-204817, filed Dec. 21, 2022, the contents of which are incorporated herein by reference.

1. A cosmetic composition comprising:
phytic acid, a salt of phytic acid, or both; and
alkoxysalicylic acid represented by a general formula (1) below, a salt of the alkoxysalicylic acid, or both, 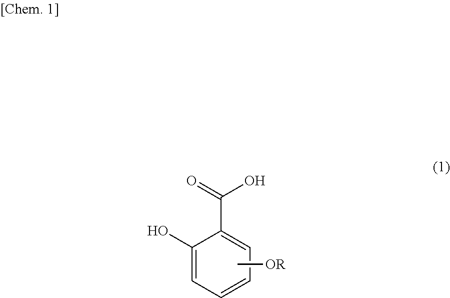
where, in the general formula (1), R is an alkyl group having a carbon number of 1 to 6.

2. The cosmetic composition according to claim 1,
wherein an amount of the phytic acid, the salt of phytic acid, or both is from 0.02 percent by mass to 0.2 percent by mass.

3. The cosmetic composition according to claim 1,
wherein R is a methyl group or an ethyl group.

4. The cosmetic composition according to claim 3,
wherein the alkoxysalicylic acid represented by the general formula (1), the salt of the alkoxysalicylic acid, or both is 4-methoxysalicylic acid.

5. A cosmetic composition for discoloration inhibition, the cosmetic composition comprising:
phytic acid, a salt of phytic acid, or both; and
alkoxysalicylic acid represented by a general formula (1) below, a salt of the alkoxysalicylic acid, or both, 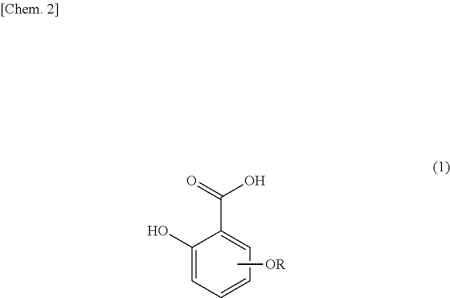
where, in the general formula (1), R is an alkyl group having a carbon number of 1 to 6.

6. A discoloration inhibitor comprising:
an active ingredient that includes phytic acid, a salt of phytic acid, or both,
wherein the discoloration inhibitor inhibits discoloration of alkoxysalicylic acid represented by a general formula (1) below, a salt of the alkoxysalicylic acid, or both, 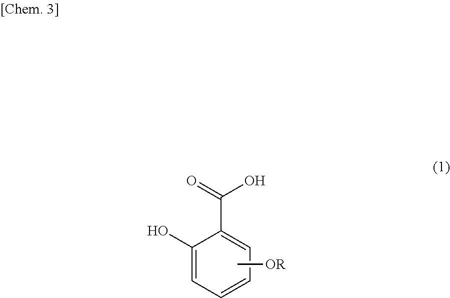
where, in the general formula (1), R is an alkyl group having a carbon number of 1 to 6.
